(19) United States
(12) Patent Application Publication
(10) Pub. No.: US 2014/0230095 A1
(43) Pub. Date: Aug. 14, 2014

(54) ANDES NEC-2

(71) Applicant: Viveros Asociados Chile Ltda. (ANA Chile-Andes Nursery Association), (US)

(72) Inventor: Rodrigo Infante, Paine (CL)

(73) Assignee: Viveros Asociados Chile Ltda. (ANA Chile-Andes Nursery Association), Paine (CL)

(21) Appl. No.: 13/766,222

(22) Filed: Feb. 13, 2013

(51) Int. Cl. A01H 5/08

(52) U.S. Cl. CPC A01H 5/0856
USPC 800/298

(57) ABSTRACT

A new and distinct variety of clingstone nectarine tree denominated ‘Andes Nec-2’ which is characterized by the fruit of which maturing a week after the ‘August Red’ nectarine that matures at the end of the harvest season. The variety has a slow pulp softening rate commencing during the last two weeks before the commercial harvest, therefore, delaying the harvest. This produces fruit with increased size and soluble solids content. The variety has a high postharvest life potential, and will maintain its initial sensory quality at low temperatures over a period of time. Its fruit is striped/solid red blush over a light yellow background color, with a round, symmetrical fruit shape.

### BACKGROUND AND SUMMARY

The present invention relates to the discovery and asexual propagation of a new variety of nectarine tree, Prunus persica denominated Andes Nec-2. This new cultivar resulted from a controlled hybridization between the nectarine Flavor Top (♀) and the nectarine genotype A67-03 (♂) performed in 1999 at Paine, Metropolitan Region, Chile (latitude −33°80′S, longitude −70°66′S, altitude 508 m above sea level).

### Distinctive Characteristics of the Variety

Semi erect plant growth, weak to medium vigor. The fruit is round, symmetrical, with a striped/solid red blush over a light yellow background color. Ripening period a week after Red August.

This variety's main feature is a slow pulp softening rate commencing during the last two weeks before the commercial harvest. This characteristic enables delaying the harvest, to increase the size of the fruit and soluble solids content without affecting their postharvest life potential. Secondly, it shows a high postharvest life potential, which can reach up to 50 days at 0° C. while maintaining its initial sensory quality. From a sensory point of view, this is a variety of medium to high acidity with a balanced sweetness/acidity ratio and in general an average sensory quality. The variety is not susceptible to “chilling injury” and therefore can be used for exporting to distant markets from the production zone.

### BRIEF DESCRIPTION OF THE FIGURES

The drawings of the nectarine of the present variety are color photographs:

FIG. 1(a) and FIG. 1(b) show two views of the whole fruit in skin color and form, and typical leaves;

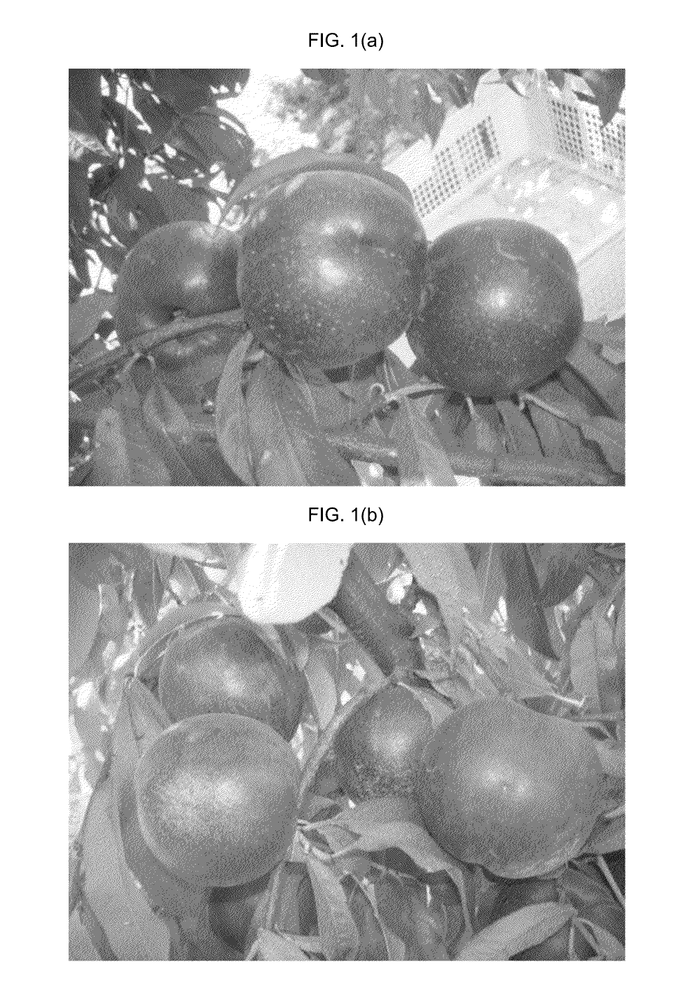

FIG. 2 shows the tree of ‘Andes Nec-2’; and

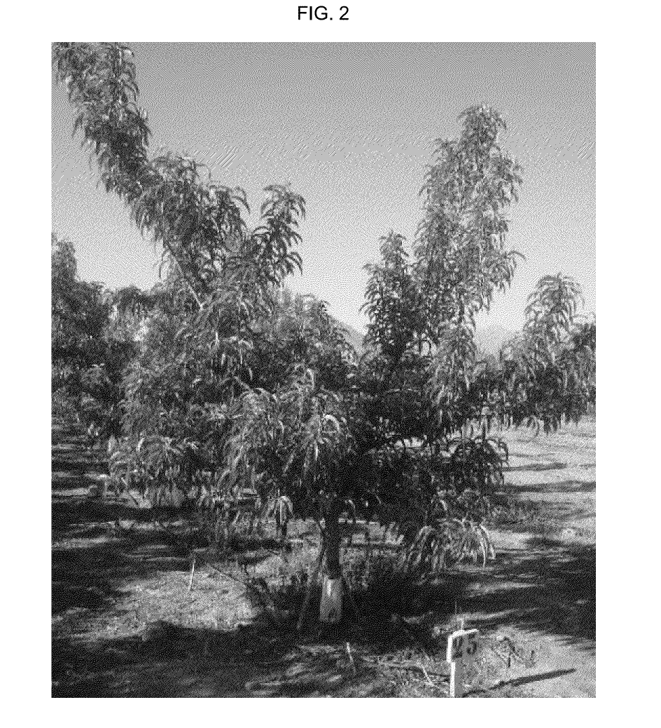

FIG. 3 shows branch of the tree of FIG. 2 and includes enlarged views of the upper surface of a leaf of ‘Andes Nec-2’.

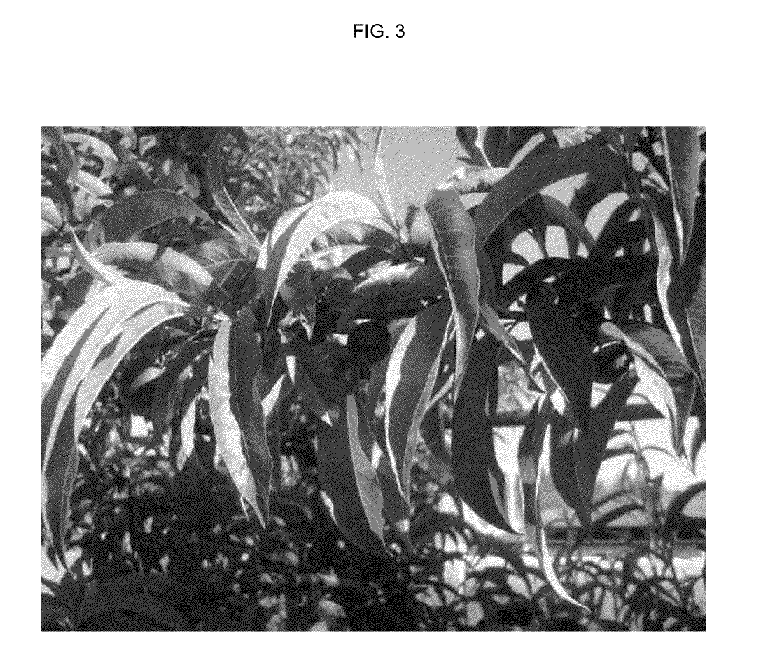

### DETAILED BOTANICAL DESCRIPTION

### Tree

1. Size: Medium, similar to Robin.
2. Vigor: Medium, similar to J. H. Hale.
3. Habit: Horizontal, like Albertina.
4. Flowering: Shoot thickness (excluding brindilles): Medium, similar to Redhaven.
5. Flowering shoot: Length of internodes: Medium, similar to Redhaven.
6. Flowering shoot: Anthocyanin coloration: Present.
7. Flowering shoot: Intensity of anthocyanin coloration (as for 6): Weak, similar to Springtime.
8. Flowering shoot: Density of flower buds (as for 4): Medium, similar Michelini.
9. Flowering shoot: General distribution of flower buds (as for 4): In groups of two or more, similar to Redhaven.

### Flower

10. Type: Showy, similar to Robin.
11. Calyx: Color of inner side (opened flower, before falling of petals): Greenish yellow, similar to Robin.
12. Corolla: Predominant color (inner side): Medium pink, similar to Fuzalode.
13. Petal: Shape: Round, similar to Springtime.
14. Petal: Size: Medium, similar to Robin.
15. Petals: Number: five, similar to Redhaven.
16. Stamens: Position compared to petals: Above, similar to Redhaven.
17. Stigma: Position compared to anthers: Above, similar to Redhaven.
18. Anthers: Pollen: Present, similar to Redhaven.
19. Ovary: Pubescence: Absent, similar to Fuzalode.
20. Young shoot: Length of stipule (fully expanded leaf): Long, similar to Robin.

### Leaf

21. Leaf blade: Length: Long.
22. Leaf blade: Width: Medium.
23. Leaf blade: Ratio length/width: Medium, similar to Early Sungrand.
24. Leaf blade: Shape in cross section: Flat, similar to Mayred.
25. Leaf blade: Recurvature of apex: Absent, similar to Merril Sundance.
26. Leaf blade: Angle at base: Acute, similar to Springtime.
27. Leaf blade: Angle at apex: Small, similar to Red June.
28. Leaf blade: Color: Green, similar to Robin.
29. Petiole: Length: Medium, similar to Genadix.
30. Petiole: Nectaries present, similar to Redhaven.
31. Petiole: Shape of nectaries: Reniform, similar to Redhaven.
32. Petiole: Predominant number of nectarines: More than two, similar to Everts.

### Fruit

33. Fruit: Size: Large, similar to Loring.
34. Fruit: Shape (in ventral view): Round, similar to Redwing.
35. Fruit: Shape of pistil end: Weakly pointed, similar to Springtime.
36. Fruit: Symmetry (viewed from pistil end): Symmetric, similar to Morettini.
37. Fruit: Prominence of suture: Weak, similar to Redhaven.
38. Fruit: Depth of stalk cavity: Medium.
39. Fruit: Width of stalk cavity: Medium.
40. Fruit: Ground color: Yellow, similar to Fuzalode.
41. Fruit: Over color present.
42. Fruit: Hue of over color: Medium red, similar to Red Diamond.
43. Fruit: Pattern of over color: Striped, similar to Verven.
44. Fruit: Extent of over color: Medium, similar to Redhaven.
45. Fruit: Pubescence: Absent.
46. Fruit: Density of pubescence: --.
47. Fruit: Thickness of skin: Medium, similar to Mme Girard.
48. Fruit: Adherence of skin to flesh: Strong, similar to Babygold5.
49. Fruit: Firmness of pulp: Firm, similar to Redhaven.
50. Fruit: Ground color of flesh: Yellow.
51. Fruit: Anthocyanin coloration directly under skin: Weakly expressed.
52. Fruit: Anthocyanin coloration of flesh: Absent.
53. Fruit: Anthocyanin coloration around stone: Strongly expressed.
54. Fruit: Texture of the flesh: Not fibrous, similar to Redhaven.
55. Fruit: Sweetness: High, similar to Philp.
56. Fruit: Acidity: High, similar to Armking.

### Stone

57. Stone: Size compared to fruit: Medium, similar to Redhaven.
58. Stone: Shape (in lateral view): Elliptical, similar to Loring.
59. Stone: Intensity of brown color: Dark.
60. Stone: Relief of surface: pits and cavites similar to Madame Girerd (4).
61 . . .
62. Stone: Tendency of splitting (at peak harvest): Absent or very low, similar to Fairhaven.
63. Stone: Adherence to flesh: Present, similar to Sweet Gold.
64. Stone: Degree of adherence to flesh: Medium, similar to Spring Crest.

### Phenology

65. Time of leaf bud burst: Early, similar to Springtime.
66. Time of beginning of flowering: Medium, similar to Redhaven.
67. Duration of flowering: Medium, similar to Redhaven.
68. Time of maturity for consumption: Late, similar to Veteran.
69. Tendency to preharvest: Absent, similar to Redhaven.

Fruit characterization

| | | Firmness (pounds) | | | | Soluble |
|---|---|---|---|---|---|---|
| | Blush | Ecuatorial | | | | solids |
| Season | (%) | Diameter | Shoulders | Suture | Tip | (° Brix) |
| 2009-2010 | 60 | 10.2 | 8.6 | 9.7 | 10.4 | 13.4 |
| 2010-2011 | 60 | 12.3 | 11.0 | 12.5 | 12.8 | 16.1 |
| 2011-2012 | 50 | 16.2 | 11.9 | 15.0 | 16.0 | 13.8 |

### Postharvest Behavior

At 50 days in cold storage +3 days at ambient temperature has good external appearance, no browning color or mealy texture, good taste and flesh firmness.

### General Technical Notes

Very good outward appearance. Excellent color coverage. Fruit round, no tip.

High productivity. Good flavor.

### CONCLUSIONS

After five years of evaluation, the variety continues to show its outstanding characteristics: very good productivity, good size, good flavor and good external appearance (some skin russeting). High potential for export purposes because of exceptional post-harvest life. having thus described and illustrated the new variety of nectarine tree, I claim:

1. A new and distinct variety of nectarine tree, substantially as illustrated and described, that is similar to ‘August Red’ nectarine by producing nectarines that mature at the end of the harvest season, yellow flesh, clingstone in type, and firm in texture, but is distinguished therefrom by maturing a week after, and by producing fruit with a better and longer postharvest potential.
